(19) United States
(12) Patent Application Publication
(10) Pub. No.: US 2023/0138787 A1
(43) Pub. Date: May 4, 2023

(54) METHOD AND APPARATUS FOR PROCESSING MEDICAL IMAGE DATA

(71) Applicant: Cygnus-Al Inc., Williston, FL (US)

(72) Inventors: Scott Anderson MIDDLEBROOKS, Beaverton, OR (US); Adrianus Cornelis KOOPMAN, BM Hilversum (NL); Ari David GOLDBERG, Chicago, IL (US); Brett Evan Edward POWELL, Roanoke, TX (US); Henricus Wilhelm VAN DER HEIJDEN, Turnhout (BE)

(73) Assignee: Cygnus-Al Inc., Williston, FL (US)

(21) Appl. No.: 17/517,986

(22) Filed: Nov. 3, 2021

(51) Int. Cl. G16H 30/40; G16H 30/20; G16H 50/20

(52) U.S. Cl. CPC G16H 30/40; G16H 30/20; G16H 50/20

(57) ABSTRACT

Disclosed herein are a method and system for processing medical image data. The method can comprise querying, using one or more monitor processors of a Picture Archiving and Communication System (PACS) monitor, a storage unit on a PACS server for available image data; determining, using the one or more monitor processors, if the available image data is new image data; retrieving, using the one or more monitor processors, the new image data from the storage unit on the PACS server if the available image data is new image data; processing, using one or more model processors, the new image data using a machine learning model to obtain a model result; generating, using the one or more model processors, at least one of an enhanced image data and a model result report based on the model result; and storing the at least one of the enhanced image data and the model result report for retrieval by a computing device.

### FIELD OF THE DISCLOSURE

The disclosure relates to computer-aided diagnosis (CAD). The disclosure also relates to a method and a platform or system for using machine learning algorithms for processing medical data. In particular, the disclosure relates to a method and apparatus for classifying nodules in medical image data.

### BACKGROUND OF THE DISCLOSURE

Advances in computed tomography (CT) allow early detection of cancer, in particular lung cancer which is one of the most common cancers. As a result, there is increased focus on using regular low-dose CT screenings to ensure early detection of the disease with improved chances of success of the following treatment. This increased focus leads to an increased workload for professionals such as radiologists who have to analyze the CT screenings.

To cope with the increased workload, computer-aided detection (CADe) and computer-aided diagnosis (CADx) systems are being developed. Hereafter both types of systems will be referred to as CAD systems. CAD systems can detect lesions (e.g. nodules) and subsequently classify them as malignant or benign. A classification need not be binary, it can also include a stage of the cancer. Usually, a classification is accompanied with a confidence value as calculated by the CAD system.

Hereafter the term “model” will be used to indicate a computational framework for performing one or more of a segmentation and a classification of imaging data. The segmentation, identification of regions of interest, and/or the classification may involve the use of a machine learning (ML) algorithm. The model comprises at least one decision function, which may be based on a machine learning algorithm, which projects the input to an output. Where the term machine learning is used, this also includes further developments such as deep (machine) learning and hierarchical learning.

Whichever type of model is used, suitable training data needs to be available to train the model. In addition, there is a need to obtain a confidence value to be able to tell how reliable a model outcome is. Most models will always give a classification, but depending on the quality of the model and the training set, the confidence of the classification may vary. It is of importance to be able to tell whether or not a classification is reliable.

While CT was used as an example in this introduction, the disclosure can also be applied to other modalities, such as ultrasound, Magnetic Resonance Imaging (MRI), Positron Emission Spectrograph (PET), Single Photon Emission Computed Tomography (SPECT), X-Ray, and the like.

### SUMMARY OF THE DISCLOSURE

It is an object of this disclosure to provide a method and apparatus for classifying nodules in imaging data.

Accordingly, the disclosed subject matter provides a computer-implemented method for processing medical image data, the method comprising:
querying, using one or more monitor processors of a Picture Archiving and Communication System (PACS) monitor, a storage unit on a PACS server for available image data;
determining, using the one or more monitor processors, if the available image data is new image data;
retrieving, using the one or more monitor processors, the new image data from the storage unit on the PACS server if the available image data is new image data;
processing, using one or more model processors, the new image data using a machine learning model to obtain a model result;
generating, using the one or more model processors, at least one of an enhanced image data and a model result report based on the model result; and
storing the at least one of the enhanced image data and the model result report for retrieval by a computing device.

In an embodiment of the disclosed subject matter, the enhanced image data is generated and the new image data and the enhanced image data are stored in the same file format, such as in a Digital Imaging and Communications in Medicine (DICOM) file format.

In an embodiment of the disclosed subject matter, the machine learning model is at least one of deep neural network, a Convolutional Neural Network (CNN or ConvNet), a U-net, a Residual Neural Network (RNN or Resnet), or a Transformer deep learning model.

In an embodiment of the disclosed subject matter, the enhanced image data is stored in the storage unit on the PACS server.

In an embodiment of the disclosed subject matter, the model result report is generated in an editable document format.

In an embodiment of the disclosed subject matter, the model result report contains text and images.

In an embodiment of the disclosed subject matter, the method further comprises storing the model result in the storage unit on the PACS server.

In an embodiment of the disclosed subject matter, generating the enhanced image data based on the model result comprises adding a visual indication to detected nodules.

The disclosed subject matter further provides a computing system comprising a Picture Archiving and Communication System (PACS) monitor including one or more monitor processors, the computing system further comprising one or more model processors for processing medical image data,

wherein the one or more monitor processors are programmed to
query a PACS server comprising a storage unit for available image data;
determine if the available image data is new image data;
retrieve the new image data from the storage unit on the PACS server if the available image data is new image data;
   wherein the one or more model processors are configured to:
process the new image data using a machine learning model to obtain a model result;
generate at least one of an enhanced image data and a model result report based on the model result; and
store the at least one of the enhanced image data and the model result report for retrieval by a computing device communicatively coupled to the PACS server.

In an embodiment of the disclosed subject matter, the enhanced image data is generated and the new image data and the enhanced image data are stored in the same file format, such as a Digital Imaging and Communications in Medicine (DICOM) file format.

In an embodiment of the disclosed subject matter, the machine learning model is at least one of deep neural network, a Convolutional Neural Network (CNN or ConvNet), a U-net, a Residual Neural Network (RNN or Resnet), or a Transformer deep learning model.

In an embodiment of the disclosed subject matter, the enhanced image data is stored in the storage unit.

In an embodiment of the disclosed subject matter, the model result report is generated in an editable document format.

In an embodiment of the disclosed subject matter, the model result report contains text and images.

In an embodiment of the disclosed subject matter, the model result is stored in the storage unit.

In an embodiment of the disclosed subject matter, the one or more processors are further programmed to generate the enhanced image data based on the model result by adding a visual indication to detected nodules.

The disclosure further provides a computer program product comprising instructions which, when executed on a processor, cause said processor to implement one of the methods or systems as described above.

### BRIEF DESCRIPTION OF THE FIGURES

Embodiments of the present disclosure will be described hereinafter, by way of example only, with reference to the accompanying drawings which are schematic in nature and therefore not necessarily drawn to scale. Furthermore, like reference signs in the drawings relate to like elements.

FIG. 1 schematically shows an overview of a workflow according to embodiments of the disclosed subject matter;

FIG. 2 schematically show a method of classifying nodules according to an embodiment of the disclosed subject matter;

FIG. 3 schematically shows a model for nodule detection according to an embodiment of the disclosed subject matter;

FIG. 4 schematically shows a system and method for processing image data according to an embodiment of the disclosed subject matter;

FIG. 5 schematically shows a method for processing image data according to an embodiment of the disclosed subject matter; and

FIG. 6 schematically shows a method for viewing image data and model results according to an embodiment of the disclosed subject matter.

FIG. 7 schematically shows a further system and method for processing image data according to an embodiment of the disclosed subject matter;

FIG. 8 schematically shows a method for processing image data according to an embodiment of the disclosed subject matter;

FIG. 9 schematically shows a method for viewing image data and model results according to an embodiment of the disclosed subject matter; and

FIG. 10 schematically shows a workstation display according to an embodiment of the disclosed subject matter.

### DETAILED DESCRIPTION

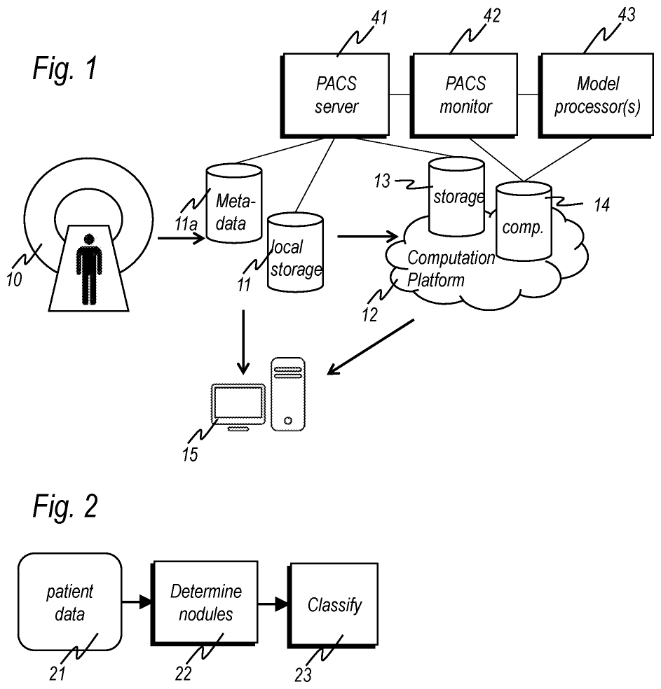

FIG. 1 schematically shows an overview of a workflow according to embodiments of the disclosed subject matter. A patient is scanned in scanning device 10. The scanning device 10 can be any type of device for generating diagnostic image data, for example an X-Ray device, a Magnetic Resonance Imaging (MRI) scanner, PET scanner, SPECT device, or any general Computed Tomography (CT) device. Of particular interest are low-dose X-Ray devices for regular and routine scans. The various types of scans can be further characterized by the use of a contrast agent, if any. The image data is typically three-dimensional (3D) data in a grid of intensity values, for example 512×512×256 intensity values in a rectangular grid.

In the following, the example of a CT device, in particular a CT device for low dose screenings, will be used. However, this is only exemplary. Aspects of the disclosure can be applied to any instantiation of imaging modality, provided that it is capable of providing imaging data. A distinct type of scan (X-Ray CT, low-dose X-Ray CT, CT with contrast agent X) can be defined as a modality.

The images generated by the CT device 10 (hereafter: imaging data) are sent to a storage 11 (step S1). The storage 11 can be a local storage, for example close to or part of the CT device 10. It can also be part of the IT infrastructure of the institute that hosts the CT device 10. The storage 11 is convenient but not essential. The data could also be sent directly from the CT device 10 to computation platform 12. The storage 11 and further database 11a can be a part of a Picture Archiving and Communication System (PACS), or they can provide data to a PACS server located elsewhere.

All or parts of the imaging data is then sent to the computation platform 12 in step S2. The computation platform 12 can comprise one or more model processors 43 for processing medical image data. The computation platform 12 can further comprise a PACS server 41 with one or more storage units and it can comprise a PACS monitor 42 with one or more monitor processors. The PACS server and/or the PACS monitor, which will be described in more detail in relation to FIG. 4, can also be located outside the computation platform (not shown in FIG. 1). The PACS server and the PACS monitor can be cloud-based or they can be dedicated (on-premise) servers. They can be located in one physical server or divided over a number of (virtual) server devices.

In general it is most useful to send all acquired data, so that the computer models of platform 12 can use all available information. However, partial data may be sent to save bandwidth, to remove redundant data, or because of limitations on what is allowed to be sent (e.g. because of patient privacy considerations). The data sent to the computation platform 12 may be provided with metadata from scanner 10, storage 11, or further database 11a. Metadata can include additional data related to the imaging data. For example statistical data of the patient (gender, age, medical history) or data concerning the equipment used (type and brand of equipment, scanning settings, etc).

Computation platform 12 comprises one or more storage devices 13 (e.g. including the PACS server 41 storage) and one or more computation devices 14 (e.g. including the PACS monitor 42 and the model processor 43), along with the necessary network infrastructure to interconnect the devices 13, 14 and to connect them with the outside world, preferably via the Internet. It should be noted that the term “computation platform” is used to indicate a convenient implementation means (e.g. via available cloud computing resources). However, embodiments of the disclosure may use a “private platform”, i.e. storage and computing devices on a restricted network, for example the local network of an institution or hospital. The term “computation platform” as used in this application does not preclude embodiments of such private implementations, nor does it exclude embodiments of centralized or distributed (cloud) computing platforms. The computation platform, or at least elements 13 and/or 14 thereof, can be part of a PACS or can be interconnected to a PACS for information exchange, in particular of medical image data.

The imaging data is stored in the storage 13. The central computing devices 14 can process the imaging data to generate feature data as input for the models. The computing devices 14 can segment imaging data. The computing devices 14 can also use the models to classify the (segmented) imaging data. More functionality of the computing devices 14 will be described in reference to the other figures.

A work station (not shown) for use by a professional, for example a radiologist, is connected to the computation platform 12. Hereafter, the terms “professional” and “user” will be used interchangeably. The work station is configured to receive data and model calculations from the computation platform. The work station can visualize received raw data and model results.

FIG. 2 schematically shows a method of classifying nodules according to an embodiment of the disclosed subject matter.

Medical image data 21 is provided to the model for nodule detection. The medical image data 21 can be 3D image data, for example a set of voxel intensities organized in a 3D grid. The medical image data can be organized into a set of slices, where each slice includes intensities on a 2D grid (say, an x-y grid) and each slice corresponds to a position along a z-axis as 3rd dimension. The data can for example be CT or MRI data. The data can have a resolution of for example 512×512×512 voxels or points.

The model for nodule detection, used in action 22 to determine nodules from the medical image data 21, may be a general deep learning model or machine learning model, in particular a deep neural network, such as a Convolutional Neural Network (CNN or ConvNet), a U-net, a Residual Neural Network (RNN or Resnet), or a Transformer deep learning model. The model can comprise a combination of said example models. The model can be trained in order to detect nodules or lesions. The model may comprise separate segmenting and classification stages, or alternatively it may segment and classify each voxel in one pass. The output of the model is a set of one or more detected nodules (assuming there is at least one or more nodules in the input data).

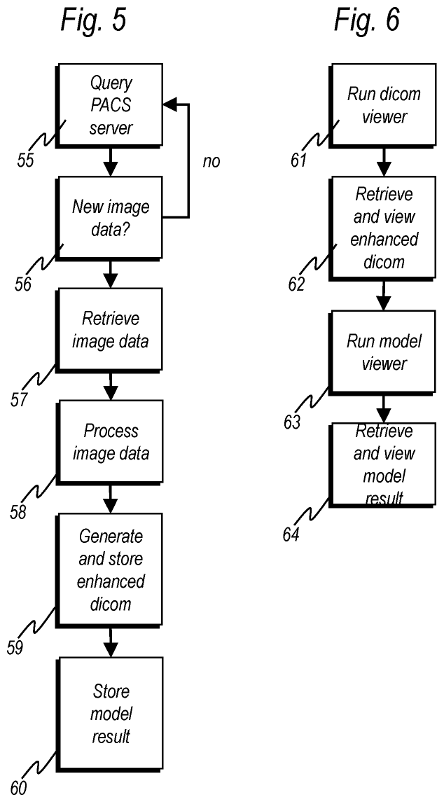

Finally, in action 23, the nodule's quality is classified based on the histogram. Further details are provided in reference to FIG. 5. The classification may be one of ground glass (also called non-solid), part solid, solid, and calcified. Based on the classification, and segmented size estimation, a lung-RADS score may be determined or at least estimated. Lung-RADS comprises a set of definitions designed to standardize lung cancer screening CT reporting and management recommendations, developed by the American College of Radiology.

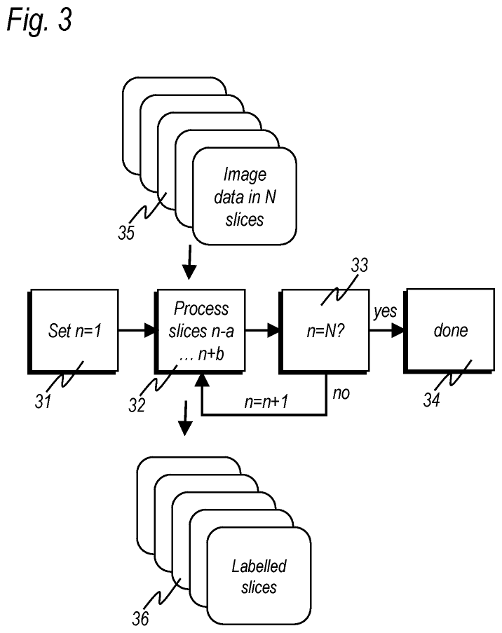

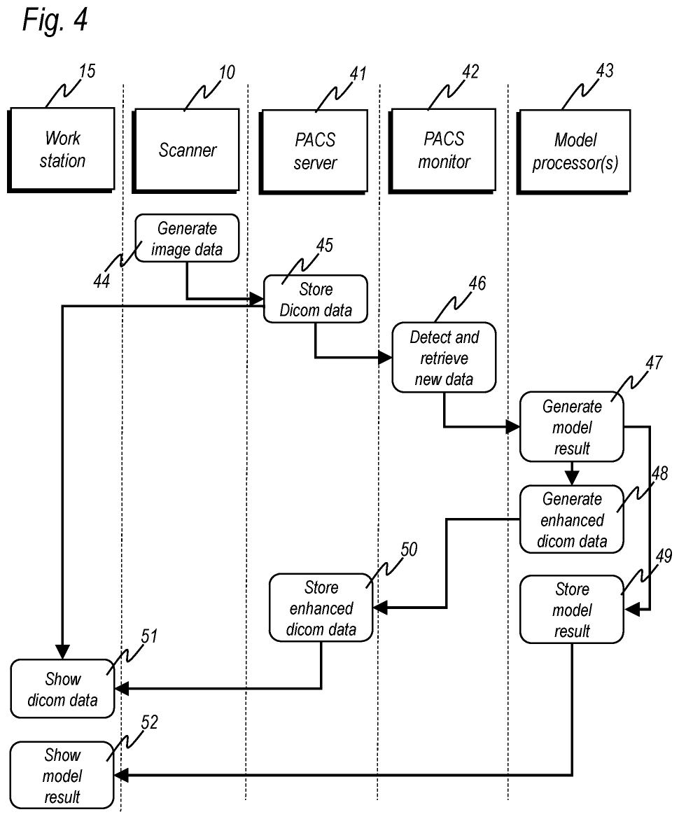

FIG. 3 schematically shows a model for nodule detection according to an embodiment of the disclosed subject matter. It is an example of how action 26 can be implemented advantageously.

The model involves an iteration over a set of N 2D image slices that together form 3D image data 35. The algorithm starts at slice n=1 (action 31) and repeats with increasing n until n=N (action 33, 34). In every iteration (action 32), a context of a+b slices n−a to n+b is evaluated. In a symmetrical processing method, a=b, so that the evaluated slice is in the middle of the data set. This is, however, not essential. Near the boundaries of the data set (n≤a or n≥b), special measures must be taken. These slices can be skipped, or data “over the boundary” can be estimated, e.g. by extrapolation or repetition of the boundary values.

As mentioned before, the prediction of the slice of data in action 32 can be done using a CNN or another machine learning model. The output is a predicted slice, where each voxel in the slice (again, possibly excluding boundary voxels) has a nodule or non-nodule label, and associated classification probability. After the full set of input slices 35 is processed, a labelled set of output slices 36 is obtained.

FIG. 4 schematically shows a system and method for processing image data according to an embodiment of the disclosed subject matter. In step 44, image data is generated by a scanner 10. The image data can be in a standard format such as DICOM (Digital Imaging and Communications in Medicine). In step 45, the image data is stored in a PACS system, for example by interacting with the Application Programming Interface (API) of a PACS server 41. The PACS server 41 includes one or more storage units for storing data.

A PACS monitor 42 is monitoring the PACS server 41. The PACS monitor can be a process on the PACS server 41 or it can run on a different computing device. The PACS monitor comprises one or more (virtual) monitor processors. The PACS monitor need not be a part of the PACS system. In step 46, the PACS monitor 42 detects that new data has been added to the PACS. In an optional step, the PACS monitor 42 determines whether or not the data is of a specific type or a specific source. For example, the PACS monitor 42 may only monitor the PACS system for image data from one or more particular scanner devices. If relevant new data is detected on the PACS system, the PACS monitor retrieves the new data and sends it to model processor 43. The model processor may be a process on a further computer system. It may also be a program that runs on the same hardware as the PACS monitor. The model processor can also be the computing device itself, e.g. a local server or a cloud-computing server.

In step 47, the one or more model processors 43 receive the new image data and process the image data using a model, such as a deep learning model. In an optional step 48, the model generates enhanced DICOM data. Enhanced DICOM data can be DICOM data wherein voxels of interest, e.g. voxels classified as belonging to a nodule, are marked by for example changing colour or contrast. The enhanced DICOM data can include additional information, such as text overlays, arrows, indicator boxes, and other graphical indicators of items of interest. The enhanced DICOM data may use a different colour scheme than standard scanner-generated DICOM data, e.g. red for regions where nodules are suspected and blue for other regions. If the enhanced DICOM data is generated, in step 50 the enhanced DICOM data may be stored on the PACS system, e.g. by using a PACS server 41 API.

In step 51, the professional can bring up the DICOM data (that was stored in step 45) on the workstation 15 for analysis. When the enhanced DICOM data is stored in the PACS system, the professional can also or instead bring up the enhanced DICOM data on the workstation 15 for analysis. Both sets of data can be viewed using a default DICOM viewer.

The model results are stored in step 49, at least temporarily. The storage can be on a persistent medium such as a hard disk drive (HDD) or solid state drive (SSD), or it can be in a non-persistent medium such as Random Access Memory (RAM).

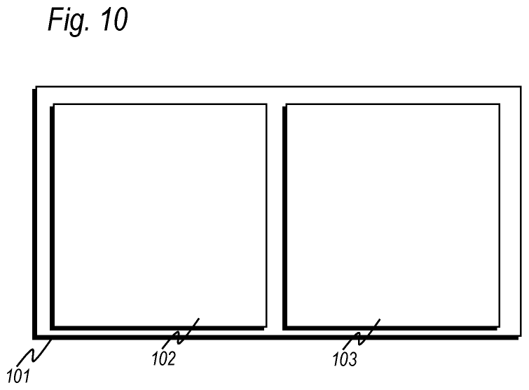

The model results can be shown on the workstation 15 of the professional, in step 52. The model results cannot be viewed with a standard PACS viewer. A dedicated model viewer program will be used to show the model result. The model viewer program typically has more options than a standard DICOM viewer. It may be able to render parts of the image data (e.g. suspected lesions) in 3D with options to look at the data from all sides. It may have colours to indicate areas of interest. It may have options to cycle through all areas of interest, in for example an order from highest interest to lowest interest. The dedicated model viewer program may display important model data, such as confidence values. The model viewer program may indicate per voxel how it is classified, e.g. lesion, tissue, bone, etc. Aspects of the model viewer program and its interaction with a standard DICOM viewer are also discussed in reference to FIG. 10.

There are various options for the model viewer program. For example, it may run as a native application on the workstation 15. It may also be a web application, so that the model viewer is a web server (for example running on the model processor 43 or a different server) that is rendered by a web browser running on the workstation 15. It may also be a different type of client-server application, with the client (thin or fat) running on the workstation 15 and communicating with a model viewer server on the workstation or on a different server. The model viewer program may also run on a different computer which renders its user interface on the workstation, for example using a virtual desktop software such as provided by Citrix or Microsoft's Remote Desktop. The model viewer program may be an X window program running on a different server but rendering on an X window system on the workstation. It may be a combination of the above approaches. In general, a skilled person will know how to present a model viewer on the workstation 15.

The professional is thus provided with two viewers for viewing the data. The standard PACS viewer can be used to view the standard DICOM data from the scanner and/or the enhanced DICOM data from the model, if that is available. In addition, the professional can view the model results using the model viewer program. In a typical usage pattern, the professional can scan the enhanced DICOM data using the standard DICOM viewer. If in the enhanced DICOM data an area is flagged as suspicious, the professional can bring up the model viewer program in order to look at the data in more detail.

FIG. 5 schematically shows the steps for generating the enhanced DICOM data and the model result data, as performed by the PACS monitor 42 and the model processor 43. First, the PACS server is queried for new image data in step 55. In step 56 it is determined if new (relevant) image data is available. If not, the flow reverts to step 55. If new image data is available, in step 57 the new image data is retrieved and processed by the model in step 58. In step 59, an DICOM data is generated based on the model results, as described in reference to FIG. 4. The model result is stored in step 60. The enhanced DICOM data is stored on the PACS server.

FIG. 6 schematically shows the steps for viewing the enhanced DICOM data on the workstation. In step 61, a standard DICOM viewer is started. The professional selects the enhanced DICOM data to be shown. In step 62, the viewer retrieves the enhanced DICOM data from the PACS server and shows it. When the professional sees something of interest in the enhanced DICOM data (e.g. a suspected region), he/she can opt to run the model viewer in step 63 on the workstation 15. In step 64, the model viewer will retrieve the model result data and show it in the model viewer. Of course, the steps 61 and 62 may be skipped by the professional, immediately proceeding to running the model viewer in step 63.

In an advantageous embodiment, the various related data sets are linked to each other, so that the professional and/or the system can be configured to easily go from one data set to another. For example, the standard (scanner-generated) DICOM data, the enhanced DICOM data and the model results all may share a same identifier (ID). The identifier may contain patient data in an anonymized manner, so that the model need not be provided with information that can identify the patient of the scan.

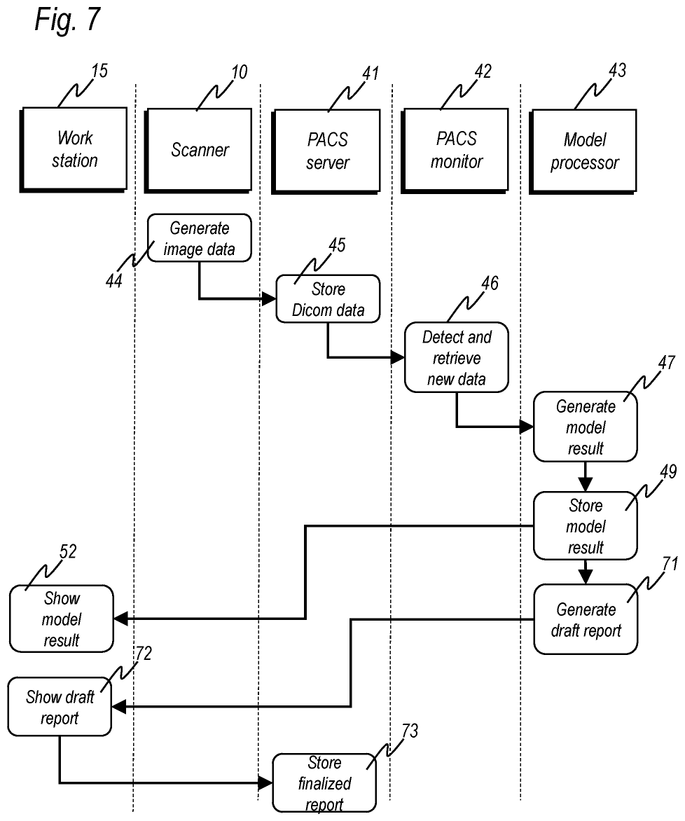

FIG. 7 schematically shows a variant of the process described in reference to FIG. 4. The different steps 71, 72, 73 may be used instead of or in addition to the steps 48, 50, 51 of FIG. 4. The steps that FIG. 7 has in common with FIG. 4 will not be described again here.

In step 71, the model processor 43 creates a model result report based on the model results. The model result report can be a draft report to be reviewed and finalized by a professional. The report can be a series of images with annotations (e.g. coloring indicating suspected regions, markings such as rectangles or circles indicating regions of interest, alternative color maps indicating voxel classifications, etc). The images can include text conveying information of relevant model results, such as type of classifications, color legend, model confidence indications, etc. The images may be 2D images similar to DICOM data, or they may be representations of 3D data generated by the model or the model viewer.

The images may be accompanied by text describing the results found. The text can include a lung-RADS score including a confidence indicator. The lung RADS score can refer to representative images which show the parts of the scan that mainly determine the lung RADS score. The text may be in the form of natural language, generated from a template or generated by a Artificial Intelligence text generator algorithm to provide a draft report for a professional to edit.

The model result report may be in any suitable digital format. It can for example be a Microsoft Word file with hi-res images included. It can be in HTML with references to image files. It can be a Portable Document Format (PDF) file, although editable file formats are preferred.

The model result report is preferably saved in draft form. It may be saved on the PACS server or it may be saved elsewhere. In step 72, the draft report is shown to the professional on the workstation 15. The professional can review the report and amend it where necessary. For example, the professional may delete images he or she deems not relevant or may edit the template or Al generated text of the report.

When the professional is satisfied that the edited or original report is up to professional standards, the report is approved and stored on the PACS server 41 in step 73. It may be preferred that only at this stage does the report become part of the official record as kept on the PACS system. This has the advantage that any false positives or other mistakes of the model do not automatically become part of the official record before they have been reviewed and corrected by a professional. In an alternative embodiment, the report is immediately stored on the PACS system. It may then be marked “draft” and “pending review” or otherwise to indicate that the report is not finalized yet. In yet another embodiment, the report is finalized and stored on the PACS system without further review and correction by the professional being necessary. The report may then still indicate a human-readable marking to state that the report is automatically generated.

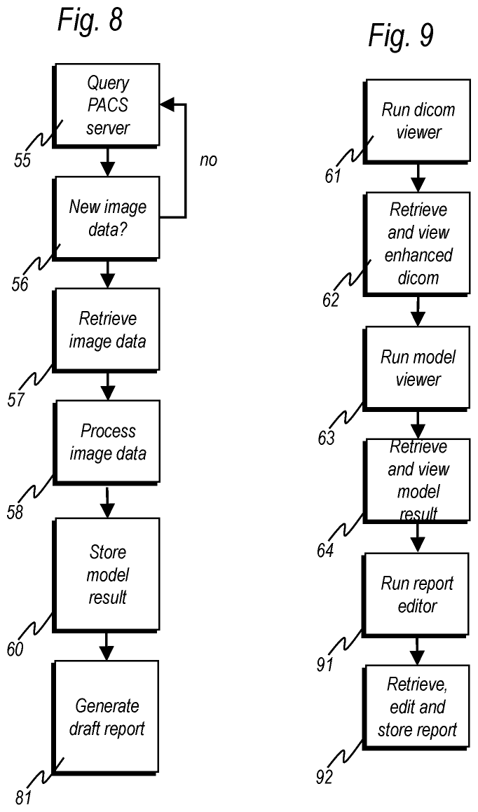

FIG. 8 schematically shows the steps performed by the PACS monitor and the model processor in the embodiment of FIG. 7. Again, the steps in FIG. 8 can be freely combined with those of FIG. 5 to form any combination thereof. Steps 55-60 have already been described and will not be repeated here. In step 81, the draft report is generated and stored as a draft. The file can be stored on the work station 15, on the PACS server 41, on the model processor 43, or elsewhere.

FIG. 9 schematically shows the steps performed on the workstation 15. Again, the steps in FIG. 8 can be freely combined with those of FIG. 6 to form any combination thereof. Steps 61-64 have already been described and will not be repeated here. In step 91, a report editor is run the workstation 15. In step 92, the draft report is retrieved from storage and shown to the professional. The professional can then edit the report and store the finalized report on the PACS system via PACS server 41.

FIG. 10 schematically shows a workstation display 101 according to an embodiment of the disclosed subject matter. In an embodiment, the professional may see the standard DICOM viewer 102 on one part of the screen, and the model viewer 103 on another. In an embodiment, the model viewer program 103 can be launched from a menu or other User Interface (UI) element of the standard DICOM viewer 102. When the model viewer program is launched in this way, it may be provided (e.g. as command line argument) with an ID for retrieving the correct model results data corresponding to the image data that is being viewed in the DICOM viewer at that time.

The standard DICOM viewer, for viewing the DICOM files or the enhanced DICOM files, need not be side by side with the model viewer program as shown in FIG. 10. They can also be arranged as tabs or in another way. It is prefered that there is a link between the applications, so that when a data set (standard or enhanced) is viewed in the DICOM viewer, the corresponding model result data set can be easily loaded in the model viewer.

In an embodiment, the model result report is shown in window 103, so that the professional can edit it while reviewing the original DICOM files in window 102. In an embodiment, the model results are shown in window 102 and the model report is shown in window 103, so that the professional can review (and if needed edit) the report while viewing the model results.

In yet another embodiment, the standard DICOM viewer, the model result viewer and the model result report editor/viewer are all shown on the workstation.

Combinations of specific features of various aspects of the disclosure may be made. An aspect of the disclosure may be further advantageously enhanced by adding a feature that was described in relation to another aspect of the disclosure.

It is to be understood that the disclosure is limited by the annexed claims and its technical equivalents only. In this document and in its claims, the verb “to comprise” and its conjugations are used in their non-limiting sense to mean that items following the word are included, without excluding items not specifically mentioned. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

1. A computer-implemented method for processing medical image data, the method comprising:
querying, using one or more monitor processors of a Picture Archiving and Communication System (PACS) monitor, a storage unit on a PACS server for available image data;
determining, using the one or more monitor processors, if the available image data is new image data;
retrieving, using the one or more monitor processors, the new image data from the storage unit on the PACS server if the available image data is new image data;
processing, using one or more model processors, the new image data using a machine learning model to obtain a model result;
generating, using the one or more model processors, at least one of an enhanced image data and a model result report based on the model result; and
storing the at least one of the enhanced image data and the model result report for retrieval by a computing device.

2. The method of claim 1, wherein the enhanced image data is generated and the new image data and the enhanced image data are stored in the same file format, such as in a Digital Imaging and Communications in Medicine (DICOM) file format.

3. The method of claim 1, wherein the machine learning model is at least one of deep neural network, a Convolutional Neural Network (CNN or ConvNet), a U-net, a Residual Neural Network (RNN or Resnet), or a Transformer deep learning model.

4. The method of claim 3, wherein the enhanced image data is stored in the storage unit on the PACS server.

5. The method of claim 1, wherein the model result report is generated in an editable document format.

6. The method of claim 5, wherein the model result report contains text and images.

7. The method of claim 1, wherein the method further comprises storing the model result in the storage unit on the PACS server.

8. The method of claim 1, wherein generating the enhanced image data based on the model result comprises adding a visual indication to detected nodules.

9. A computing system comprising a Picture Archiving and Communication System (PACS) monitor including one or more monitor processors, the computing system further comprising one or more model processors for processing medical image data,
wherein the one or more monitor processors are programmed to
query a PACS server comprising a storage unit for available image data;
determine if the available image data is new image data;
retrieve the new image data from the storage unit on the PACS server if the available image data is new image data;
wherein the one or more model processors are configured to:
process the new image data using a machine learning model to obtain a model result;
generate at least one of an enhanced image data and a model result report based on the model result; and
store the at least one of the enhanced image data and the model result report for retrieval by a computing device communicatively coupled to the PACS server.

10. The system of claim 9, wherein the enhanced image data is generated and the new image data and the enhanced image data are stored in the same file format, such as a Digital Imaging and Communications in Medicine (DICOM) file format.

11. The system of claim 9, wherein the machine learning model is at least one of deep neural network, a Convolutional Neural Network (CNN or ConvNet), a U-net, a Residual Neural Network (RNN or Resnet), or a Transformer deep learning model.

12. The method of claim 11, wherein the enhanced image data is stored in the storage unit.

13. The system of claim 9, wherein the model result report is generated in an editable document format.

14. The system of claim 13, wherein the model result report contains text and images.

15. The system of claim 9, wherein the model result is stored in the storage unit.

16. The system of claim 9, wherein the one or more processors are further programmed to generate the enhanced image data based on the model result by adding a visual indication to detected nodules.
